(19) United States
(12) Patent Application Publication
(10) Pub. No.: US 2010/0267682 A1
(43) Pub. Date: Oct. 21, 2010

(54) CORTICOSTEROIDS TO TREAT EPOTHILONE OR EPOTHILONE DERIVATIVE INDUCED DIARRHEA

(75) Inventors: Anandhi Ranganathan Johri, Short Hills, NJ (US); Paul M.J. McSheehy, Loerrach-Steffen (DE); Dirk Weber, Allschwil (CH)

Correspondence Address: NOVARTIS;CORPORATE INTELLECTUAL PROPERTY, ONE HEALTH PLAZA 101/2, EAST HANOVER, NJ, 07936-1080, US

(73) Assignee: NOVARTIS AG, Basel (CH)

(21) Appl. No.: 12/741,680

(22) PCT Filed: Oct. 15, 2008
(86) PCT No.: PCT/US08/79936

§ 371 Date: May 6, 2010

Related U.S. Application Data
(60) Provisional application No. 60/986,635, filed on Nov. 9, 2007.

(51) Int. Cl. A61K 31/573; A61K 31/427; A61P 1/12

(52) U.S. Cl. 514/171; 514/369

(57) ABSTRACT

Epothilone derivatives are co-administered with an antidiarrheal agent, e.g., a corticosteroid, or a glucocorticoid steroid in the treatment of a proliferative disease.

The invention relates to corticosteroids as antidiarrheal agents to treat diarrhea induced by epothilone or derivatives of epothilone. The present invention also relates to pharmaceutical combinations comprising: (a) corticosteriods, such a glucocorticoid steroids; and (b) an epothilone derivative of formula (I), and optionally at least one pharmaceutically acceptable carrier for simultaneous, separate or sequential use, in particular, for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The microtubule-stabilizing effect of epothilones was first described by Bollag et al., Cancer Res, Vol. 55, pp. 2325-33 (1995). A suitable treatment schedule of different types of tumors, especially tumors which are refractory to the treatment by other chemotherapeutics, in particular, TAXOL™, is described in WO 99/43320.

The present invention pertains to a combination, such as a combined preparation or a pharmaceutical composition, which comprises: (a) a corticosteroid and (b) an epothilone derivative of formula (I)

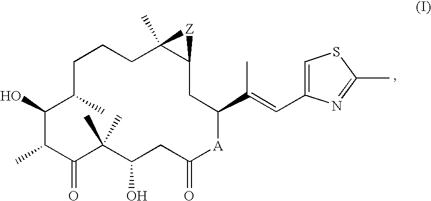

in which
compound A represents O or NR_{N}, wherein R_{N} is hydrogen or lower alkyl;
R is hydrogen or lower alkyl; and
Z is O or a bond, in which the active ingredients (a) and (b) are present in each case in free form or in the form of a pharmaceutically acceptable salt and optionally at least one pharmaceutically acceptable carrier; for simultaneous, separate or sequential use.

A compound of formula (I), wherein A represents O, R is hydrogen and Z is O is known as epothilone A; a compound of formula (I), wherein A represents O, R is methyl and Z is O is known as epothilone B; a compound of formula (I), wherein A represents O, R is hydrogen and Z is a bond is known as epothilone C; a compound of formula (I), wherein A represents O, R is methyl and Z is a bond is known as epothilone D.

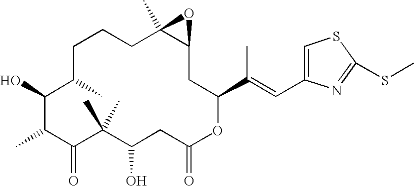

The term “a combined preparation”, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of the diarrhea that the patient experiences.

The present invention especially relates to a combined preparation, which comprises: (a) one or more unit dosage forms of a corticosteroid antidiarrheal agent; and (b) one or more unit dosage forms of an epothilone derivative of formula (I), especially epothilone B. The present invention further relates to a combined preparation, which comprises: (a) one or more unit dosage forms of a glucocorticosteroid antidiarrheal agent; and (b) one or more unit dosage forms of an epothilone derivative of formula (I), especially epothilone B. The present invention especially relates to a combined preparation, which comprises: (a) one or more unit dosage forms of a corticosteroid antidiarrheal agent selected from the group consisting of prednisone, prednisolone and dexamethosone; and (b) one or more unit dosage forms of an epothilone derivative of formula (I), especially epothilone B.

The antidiarrheal agent is a corticosteroid, such as a glucocorticoid steriod that includes, but is not limited to, prednisone, prednisolone and dexamethosone, that is administered to prevent, control or eliminate diarrhea that is sometimes associated with the administration of epothilones, especially epothilone B. Thus, the present invention also relates to a method of preventing or controlling diarrhea associated with administering an epothilone derivative of formula (I), which comprises administering an effective amount of an corticosteroid antidiarrheal agent to the patient receiving treatment with the epothilone derivative. The present invention also relates to a method of preventing or controlling diarrhea associated with administering an epothilone derivative of formula (I), which comprises administering an effective amount of an glucocorticosteroid antidiarrheal agent selected to the patient receiving treatment with the epothilone derivative. The present invention especially relates to a method of preventing or controlling diarrhea associated with administering an epothilone derivative of formula (I), which comprises administering an effective amount of an corticosteroid antidiarrheal agent selected from predinsone, prednisolone, and dexamethosone to the patient receiving treatment with the epothilone derivative.

Corticosteroids are a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels and behavior.

Glucocorticoids are a class of steroid hormones characterized by an ability to bind with the cortisol receptor.

Glucocorticoids, such as cortisol control carbohydrate, fat and protein metabolism and are anti-inflammatory by preventing phospholipid release, decreasing eosinophil action and a number of other mechanisms. Synthetic versions are dexamethosone and prednisone.

Corticosteroids and glucocorticoid steroids are described in the Handbook of Cancer Chemotherapy 6th Ed. R T Skeel; 2003 Lippincott Williams & Wilkins and the Review of Medical Physiology 8^{th} Ed, W F Ganong; 1977 Lange Medical Publications.

The term “solid tumor” especially means breast cancer, ovarian cancer, cancer of the colon and generally the GI tract, cervix cancer, lung cancer, in particular small-cell lung cancer, and non-small-cell lung cancer, head and neck cancer, bladder cancer, cancer of the prostate or Kaposi's sarcoma, especially colorectal cancer, ovarian cancer, and prostate cancer. The present combination inhibits the growth of solid tumors, but also liquid tumors. Furthermore, depending on the tumor type and the particular combination used a decrease of the tumor volume can be obtained.

Thus, the present invention also relates to a method of preventing or controlling diarrhea associated with administering an epothilone derivative of formula (I) to a patient suffering from a solid tumor disease, which comprises administering an effective amount of an corticosteroid antidiarrheal agent to the patient receiving treatment with the epothilone derivative. The present invention further relates to a method of preventing or controlling diarrhea associated with administering an epothilone derivative of formula (I) to a patient suffering from a solid tumor disease selected from breast cancer, ovarian cancer, cancer of the colon, and generally the GI tract, cervix cancer, lung cancer, in particular small-cell lung cancer, and non-small-cell lung cancer, head and neck cancer, bladder cancer, cancer of the prostate or Kaposi's sarcoma, which comprises administering an effective amount of an corticosteroid antidiarrheal agent to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, ovarian cancer, and prostate cancer, which comprises administering an effective amount of an corticosteroid antidiarrheal agent to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, which comprises administering an effective amount of a corticosteroid antidiarrheal agent to the patient receiving treatment with the epothilone derivative.

Thus, the present invention also relates to a method of preventing or controlling diarrhea associated with administering an epothilone derivative of formula (I) to a patient suffering from a solid tumor disease, which comprises administering an effective amount of an glucocorticosteroid antidiarrheal agent to the patient receiving treatment with the epothilone derivative. The present invention further relates to a method of preventing or controlling diarrhea associated with administering an epothilone derivative of formula (I) to a patient suffering from a solid tumor disease selected from breast cancer, ovarian cancer, cancer of the colon, and generally the GI tract, cervix cancer, lung cancer, in particular small-cell lung cancer, and non-small-cell lung cancer, head and neck cancer, bladder cancer, cancer of the prostate or Kaposi's sarcoma, which comprises administering an effective amount of a glucocorticosteroid antidiarrheal agent to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, ovarian cancer, and prostate cancer, which comprises administering an effective amount of an glucocorticosteroid antidiarrheal agent to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, which comprises administering an effective amount of a glucocorticosteroid antidiarrheal agent to the patient receiving treatment with the epothilone derivative.

Thus, the present invention also relates to a method of preventing or controlling diarrhea associated with administering an epothilone derivative of formula (I) to a patient suffering from a solid tumor disease, which comprises administering an effective amount of an corticosteroid antidiarrheal agent selected from prednisone, prednisolone and dexomethosone, to the patient receiving treatment with the epothilone derivative. The present invention further relates to a method of preventing or controlling diarrhea associated with administering an epothilone derivative of formula (I) to a patient suffering from a solid tumor disease selected from breast cancer, ovarian cancer, cancer of the colon, and generally the GI tract, cervix cancer, lung cancer, in particular small-cell lung cancer, and non-small-cell lung cancer, head and neck cancer, bladder cancer, cancer of the prostate or Kaposi's sarcoma, which comprises administering an effective amount of an corticosteroid antidiarrheal agent selected from prednisone, prednisolone and dexomethosone, to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, ovarian cancer, and prostate cancer, which comprises administering an effective amount of an corticosteroid antidiarrheal agent selected from prednisone, prednisolone and dexomethosone, to the patient receiving treatment with the epothilone derivative. The present invention especially relates to a method of preventing or controlling diarrhea associated with administering an epothilone derivative of formula (I) to a patient suffering from a solid tumor disease selected from colorectal cancer, which comprises administering an effective amount of a corticosteroid antidiarrheal agent selected from prednisone, prednisolone and dexomethosone, to the patient receiving treatment with the epothilone derivative.

The structure of the active agents identified by code nos., generic or trade names may be taken from the actual edition of the standard compendium “The Merck Index” or from databases, e.g., Patents International (e.g., IMS World Publications). The corresponding content thereof is hereby incorporated by reference.

It will be understood that references to the combination partners (a) and (b) are meant to also include the pharmaceutically acceptable salts. If these combination partners (a) and (b) have, e.g., at least one basic center, they can form acid addition salts. Corresponding acid addition salts can also be formed having, if desired, an additionally present basic center. The combination partners (a) and (b) having an acid group (e.g., COOH) can also form salts with bases. The combination partner (a) or (b) or a pharmaceutically acceptable salt thereof may also be used in form of a hydrate or include other solvents used for crystallization.

Epothilone derivatives of formula (I), wherein A represents O or NR_{N}, wherein R_{N} is hydrogen or lower alkyl, R is hydrogen or lower alkyl and Z is O or a bond, and methods for the preparation of such epothilone derivatives are in particular generically and specifically disclosed in the patents and patent applications WO 93/10121, U.S. Pat. No. 6,194,181, WO 98/25929, WO 98/08849, WO 99/43653, WO 98/22461 and WO 00/31247 in each case, in particular, in the compound claims and the final products of the working examples, the subject-matter of the final products, the pharmaceutical preparations and the claims is hereby incorporated into the present application by reference to this publications. Other aspects of the invention include epothilone derivatives disclosed in the patents and patent applications WO 93/10121, U.S. Pat. No. 6,194,181, WO 98/25929, WO 98/08849, WO 99/43653, WO 98/22461 and WO 00/31247 used with antidiarreal agents of the present invention. Comprised are likewise the corresponding stereoisomers as well as the corresponding crystal modifications, e.g., solvates and polymorphs, which are disclosed therein.

The transformation of epothilone B to the corresponding lactam is disclosed in Scheme 21 (page 31, 32) and Example 3 of WO 99/02514 (pages 48-50). The transformation of a compound of formula (I) which is different from epothilone B into the corresponding lactam can be accomplished analogously. Corresponding epothilone derivatives of formula (I), wherein R_{N} is lower alkyl can be prepared by methods known in the art, such as a reductive alkylation reaction starting from the epothilone derivative wherein R_{N} is hydrogen.

Epothilone derivatives of formula (I), especially epothilone B (patupilone), can be administered as part of pharmaceutical compositions which are disclosed in WO 99/39694.

In a specific embodiment, the epothilone derivative is a compound of formula (I), in which compound A represents O or NR_{N}, wherein R_{N} is hydrogen or lower alkyl, R is hydrogen or lower alkyl, and Z is O or a bond.

In the epothilone derivative of formula (I) preferably A represents O, R is lower alkyl, e.g., ethyl or, most preferably, methyl and Z is preferably O.

Unless stated otherwise in the present disclosure organic radicals designated “lower” contain not more than 7, preferably not more than 4, carbon atoms and the following expressions have the meanings as given below:

The present invention especially relates to glucocorticoids, such as prednisolone, as antidiarrheal agents to treat diarrhea induced by one or more unit dosage forms of an epothilone derivative of formula (I), especially epithilone B.

As understood by one skilled in the art, the treatment of diarrhea includes but is not limited to the prevention and/or control and/or eliminate diarrhea.

A combination which comprises: (a) glucocorticoid, such as prednisolone, as antidiarrheal agents; and (b) an epothilone derivative of formula (I), in which compound A represents O or NR_{N}, wherein R_{N} is hydrogen or lower alkyl, R is hydrogen or lower alkyl, and Z is O or a bond, in which the active ingredients are present in each case in free form or in the form of a pharmaceutically acceptable salt and optionally at least one pharmaceutically acceptable carrier, will be referred to hereinafter as a COMBINATION OF THE INVENTION.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

The antidiarrheal agent is administered as a preventative measure throughout the cycle or as needed when diarrhea occurs.

In another embodiment of the invention the antidiarrheal agent is prednisolone.

In another embodiment of the present invention, the subject receives the epothilone derivative of formula (I) once weekly for several weeks, e.g., three weeks, followed by one or several weeks off and the antidiarrheal agent is administered as a preventative measure by pretreating the subject with the antidiarrheal agent before the administration of the epothilone derivative begins and continuing administration of the antidiarrheal agent throughout the cycles, or by administering the antidiarrheal agent throughout the cycles without pretreatment or by administering antidiarrheal agent as needed when diarrhea occurs during the cycles, with or without a pretreatment. As an example, when the epothilone derivative is administered once weekly for three weeks with one week off, each four week interval will be considered one cycle.

An effective amount of the antidiarrheal agent is an amount sufficient to treat diarrhea such as to prevent, control, or eliminate diarrhea associated with the administration of an epothilone derivative, especially it is an amount which increases the amount of the epothilone derivative that can administered when the diarrhea is the does limiting toxicity of the epothilone derivative, especially epothilone B.

The COMBINATION OF THE INVENTION can be a combined preparation or a pharmaceutical composition.

The pharmaceutical compositions according to the invention can be prepared in a manner known per se and are those suitable for enteral, such as oral or rectal, and parenteral administration to mammals (warm-blooded animals), including man.

The novel pharmaceutical composition contain, e.g., from about 10% to about 100%, preferably from about 20% to about 60%, of the active ingredients. Pharmaceutical preparations for the combination therapy for enteral or parenteral administration are, e.g., those in unit dosage forms, such as sugar-coated tablets, tablets, capsules or suppositories, and furthermore ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, e.g., water, glycols, oils, alcohols, flavoring agents, preservatives, coloring agents; or carriers such as starches, sugars, microcristalline cellulose, diluents, granulating agents, lubricants, binders, disintegrating agents and the like in the case of oral solid preparations, such as, e.g., powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partner of the COMBINATION OF THE INVENTION may be administered separately, i.e., the components may be administered simultaneously or sequentially and in any order. For example, the method of treatment of a proliferative disease according to the invention may comprise: (i) administration of the first combination partner in free or pharmaceutically acceptable salt form; and (ii) administration of the second combination partner in free or pharmaceutically acceptable salt form, simultaneously or sequentially in any order, in jointly therapeutically effective amounts, preferably in synergistically effective amounts, e.g., in daily dosages corresponding to the amounts described herein. The individual combination partners of the COMBINATION OF THE INVENTION can be administered separately at different times during the course of therapy or concurrently in divided or single combination forms. One agents can, e.g., be an enteral formulation and the other can be administered parenterally. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the epothilone derivative within the range that yields efficacy without toxicity requires a regimen based on the kinetics of the active ingredients' availability to target sites. This involves a consideration of the distribution, equilibrium and elimination of the active ingredients.

If the warm-blooded animal is a human or patient a patient in need thereof of an effective amount of a compound of formula (I) and an antidiarrheal agent, the dosage of a compound of formula (I) is preferably in the range of about 0.25 to 75, preferably 0.5 to 50, e.g., 2.5, mg/m² once weekly for two to four, e.g., three, weeks, followed by 6 to 8 days off in the case of an adult patient.

The antidiarrheal agent is preferably administered from one or twice per day according to established protocols for the antidiarrheal agent. The dosage of an antidiarrheal agent can be from about 1 to about 100 mg/m² per day. This dosage of the antidiarrheal agent can be administered from about 3 to about or up to 7 days. The treatment days may be, but not limited, to consecutive days.

Moreover, the present invention relates to a method of treating a warm-blooded animal having a proliferative disease comprising administering to the animal a COMBINATION OF THE INVENTION in a quantity which is therapeutically effective against a proliferative disease and which reduces any diarrhea associated with the administration of the epothilone derivative.

Furthermore, the present invention pertains to the use of a COMBINATION OF THE INVENTION for the treatment of a proliferative disease and for the preparation of a medicament for the treatment of a proliferative disease.

Moreover, the present invention provides a commercial package comprising as active ingredients COMBINATION OF THE INVENTION, together with instructions for simultaneous, separate or sequential use thereof in the treatment of a proliferative disease.

### EXAMPLES

The following Examples illustrate the invention described above; they are not, however, intended to limit the scope of the invention in any way. The beneficial effects of the COMBINATION OF THE INVENTION can also be determined by other test models known as such to the person skilled in the pertinent art.

### BDix Rat Tumor Model

Rats: Female BDix rats weighing at least 150 g are used for the experiments. They are idendified by ear markings and kept in groups of 4 under normal conditions with access to food and water as libitum.
Tumors: Rat glioma cells, A15 (aks 1a2r) are obtained from ECACC. They are cultivated in DMEM medium with the addition of 2 mM glutamine and 15% FBS. Tumors are induced by concentrating 5×10⁵ cells in 50 μl and injecting s.c. in the flank of the rats. Tumors are visible 1 week post inoculation and are measured using calipers by applying the formula l*w*h*π/6. Generally experiments begin when the tumors are >150 mm³.
Patupilone: Patupilone is weighed and dissolved in a vehicle of 30% PEG-300 and 70% normal saline to a final concentration of 1 mg/ml. Patupilone is injected i.v. in the tail vein as a bolus of 2-3 seconds at a dose of 1.5 mg/kg once.
Prednisolone: Prednisolone tablets (20 mg) are ground up in a 0.5% methylcellulose solution added to 0.025% Tween-20 detergent. The suspension is sonicated for 30 min to achieve a milky suspension for oral administration (5 mL/kg).

Although prednisolone is administered orally daily, the dose and number of days of treatment are different in each experiment. The doses are 1, 3, 7 or 10 mg/kg which is roughly equivalent to 6, 17, 40 and 60 mg/m², respectively. When prednisolone and patupilone treatment are on the same day, prednisolone is administered 2-4 hours after treatment with patupilone.

Prednisolone is a corticosteroid and is the product of normal liver metabolism (hydroxylation) of prednisone.

Diarrhea: Diarrhea is monitored daily from day 3 post injection fo patupilone which is on day 0. Diarrhea is scored as follows:

Grade 0: normal stool

### Grade 0.5

Grade 1: soft but formed stool

### Grade 1.5

Grade 2: soft unformed stool

### Grade 2.5

Grade 3: liquid stool

Thus, each rat on each day of diarrhea (5 days from day 3 to 5) is graded 0-3 which allows a semi-quantitative analysis of the effects of different treatment. Two different abalytical methods are applied using a) area under the curve (AUC_{days3-7}) calculated using the trapezoidal rule as applied by Graphpad Prism (v. 4.0 for Windows) and b) the numbere of days in which diarrhea is graded >1. It is believed that the second approach is a more realistic comparison to the clinic since as described above G≦1 is not really diarrhea, and in the clinic, the major issue is avoiding severe diarrhea, i.e. clinical grades of ≧2.

Efficacy and Tolerability: Tumor volume (Tvol) is quantified by change in tumor volume (endpoint vs. starting value in mm³). As the T/C_{TVol}, i.e. [ΔTVOl_{drug}/ΔTVol_{vehicle}]. The body weight of the rats is measured three times per week.

### Example 1

BDix rats (180 g) are treated once with patupilone (PAT) at 1.5 mg/kg i.v. bolus and on the same day and for 6 further days with prednisolone (Predn) at 0, 1, 3 or 10 mg/kg p.o. Results show the mean±SEM for diarrhea, survival and body-weight (BW), where *P<0.05, **P<0.01, ***P<0.001 versus the control i.e. rats receiving patupilone alone with the prednisolone vehicle (one-way ANOVA with Tukey applied post-hoc).

| | Host Response | | | | | |
|---|---|---|---|---|---|---|
| | | | | | ΔBW | |
| | Diarrhoea: | Diarrhoea: | | BW (g) | (g) | Δ% BW |
| Treatment | AUC | Days > G1 | Survival | Day-7 | Day-7 | Day-7 |
| PAT alone | 7.0 ± 0.7 | 2.7 ± 0.2 | 6/6 | 150 ± 3 | −29.5 ± 3.1 | −16.3 ± 1.3 |
| & Predn | | | | | | |
| vehicle | | | | | | |
| PAT & | 5.9 ± 0.7 | 2.2 ± 0.3 | 6/6 | 142 ± 5 | −38.0 ± 2.2 | −21.2 ± 1.2* |
| 1 mg/kg | | | | | | |
| Predn | | | | | | |
| PAT & 3 mg/kg | 2.1 ± 0.6*** | 0.3 ± 0.2*** | 6/6 | 141 ± 4 | −38.3 ± 2.0 | −21.3 ± 0.6* |
| Predn | | | | | | |
| PAT & | 1.4 ± 0.6*** | 0.2 ± 0.2 | 6/6 | 139 ± 4 | −40.7 ± 3.0* | −22.6 ± 1.3* |
| 10 mg/kg | | | | | | |
| Predn | | | | | | |

### Example 2

Rat A15 glioma cells are injected s.c. into BDix rats (190 g) which after 10 days are treated once with patupilone (PAT) at 1.5 mg/kg i.v. bolus and on the same day and for 6 further days with prednisolone (Predn) at 0, 3 or 10 mg/kg p.o. Results show the mean±SEM for diarrhea, survival and body-weight (BW), where *P<0.05, versus the control i.e. rats receiving patupilone alone with the prednisolone vehicle (one-way ANOVA with Tukey applied post-hoc).

| | Host Response | | | | | |
|---|---|---|---|---|---|---|
| | Tumour | Diarrh: | Diarrh: | | BW (g) | Δ% BW |
| Treatment | Wt (g) | AUC | Days > G1 | Survival | Day-6 | Day-13 |
| PAT alone & | 0.83 ± 0.17 | 4.0 ± 0.5 | 1.7 ± 0.3 | 6/6 | 186 ± 3 | −4.1 ± 2.1 |
| Predn vehicle | | | | | | |
| PAT & 3 mg/kg | 0.75 ± 0.12 | 0.5 ± 1.2 | 0 ± 0* | 6/6 | 169 ± 11 | −12.7 ± 4.4 |
| Predn | | | | | | |
| PAT & 10 mg/kg | 0.88 ± 0.32 | 0.1 ± 0.1* | 0 ± 0* | 6/6 | 183 ± 3 | −2.4 ± 2.1 |
| Predn | | | | | | |

### Example 3

Rat A15 glioma cells are injected s.c. into BDix rats (185-195 g). After 10 days the rats are treated on day-0 once with patupilone (PAT) at 1.5 mg/kg i.v. bolus (G2, G4-G7) or PAT-vehicle (G1 & G3). All rats treated with prednisolone (Predn) received the drug p.o. at 7 mg/kg for 5 consecutive days (except G7 for 3 days only) either 1-day before (G4), on the same day (G7), 1-day after patupilone (G6) or 3-days after patupilone (G5). Results show the mean±SEM for tumour volume, diarrhea, survival and body-weight (BW), where *P<0.05, **P<0.01, ***P<0.001 versus the relevant control i.e. vehicles (G1) for TVol and BW (one-way ANOVA with Tukey applied post-hoc) or rats receiving patupilone alone & Predn-Vehicle (G2) for diarrhea (two-way ANOVA with Tukey applied post-hoc). #Three rats are culled on day-7 due to BW-loss >20% and morbidities.

| | | | | | Host Response | | | | | |
|---|---|---|---|---|---|---|---|---|---|---|
| | | Tumour response | | | | | | BW | | |
| | | TVol (g) | ΔTVol□ | T/C | Diarrh: | Diarrh: | | (g) | Δ% BW | Δ% BW |
| Treatment | | Day-14 | Day-14 | Day-14 | AUC | Days > G1 | Survival | Day-14 | Day-7 | Day-14 |
| G1 | Vehicles | 934 ± 173 | 785 ± 169 | 1.0 | 0 | 0 | 8/8 | 199 ± 7 | 0.3 ± 2.0 | 8.4 ± 1.9 |
| G2 | PAT | 426 ± 47 | 277 ± 37 | 0.35* | 6.8 ± 0.7 | 2.9 ± 0.3 | 8/8 | 179 ± 4 | −18.6 ± 1.3*** | −4.4 ± 1.8*** |
| | alone & | | | | | | | | | |
| | Predn | | | | | | | | | |
| | vehicle | | | | | | | | | |
| G3 | Predn | 877 ± 103 | 728 ± 93 | 0.93 | 0 | 0 | 8/8 | 207 ± 6 | 3.7 ± 1.1 | 10.5 ± 1.5 |
| | alone & | | | | | | | | | |
| | PAT | | | | | | | | | |
| | vehicle | | | | | | | | | |
| G4 | PAT & | 432 ± 38 | 288 ± 45 | 0.37 | 4.2 ± 0.8 | 1.9 ± 0.4*** | 8/8 | 179 ± 4 | −23.3 ± 0.8*** | −5.9 ± 1.1*** |
| | Predn | | | | | | | | | |
| | Day-1 | | | | | | | | | |
| G5 | PAT & | 367 ± 32 | 219 ± 35 | 0.28* | 4.1 ± 0.5 | 1.6 ± 0.3*** | 8/8 | 180 ± 6 | −22.0 ± 1.1*** | −3.2 ± 1.5*** |
| | Predn | | | | | | | | | |
| | Day 3 | | | | | | | | | |
| G6 | PAT & | 352 ± 37 | 203 ± 27 | 0.26* | 3.9 ± 0.6* | 1.5 ± 0.3*** | 8/8 | 175 ± 6 | −23.4 ± 1.4*** | −7.5 ± 2.2*** |
| | Predn | | | | | | | | | |
| | Day 1 | | | | | | | | | |
| G7 | PAT & | 475 ± 57 | 320 ± 52 | 0.41 | 5.1 ± 0.8 | 1.6 ± 0.3** | 5/8 # | 180 ± 5 | −19.6 ± 2.5*** | −3.9 ± 2.6** |
| | Predn | | | | | | | | | |
| | Day 0 | | | | | | | | | |
| | (for 3 | | | | | | | | | |
| | days) | | | | | | | | | |

### Example 4

Rat A15 glioma cells are injected s.c. into BDix rats (185-195 g) and after 10 days both groups are treated on day-0 once with patupilone (PAT) at 1.5 mg/kg i.v. bolus (G2, G4). G4 received prednisolone (Predn) p.o. at 7 mg/kg for 5 consecutive days while G2 received Predn-vehicle only for 5 days. Results show the mean±SD (3 rats for each group at each timepoint) for the total PAT exposure (AUC) in each matrix; the mean difference is determined as the T/C (i.e. G4-value/G2-value) and the associated P-value is from a 2-tailed t-test.

| | AUC_{24-336 hr} (mean ± SD, n = 3) ng/hr/mL | | | |
|---|---|---|---|---|
| | G2: PAT & Veh- | | | |
| Matrix | Predn | G4: PAT & Predn D-1 | T/C | P-value |
| Blood | 1891 ± 344 | 2477 ± 171 | 1.31 | 0.057 |
| Brain | 64948 ± 6237 | 56713 ± 14461 | 0.87 | 0.42 |
| Caecum | 53911 ± 10512 | 59882 ± 10885 | 1.11 | 0.53 |
| Jejunum | 57045 ± 3988 | 53150 ± 7655 | 0.93 | 0.48 |
| Liver | 56278 ± 9988 | 44454 ± 13379 | 0.79 | 0.29 |
| Tumor | 142437 ± 36429 | 139950 ± 14245 | 0.98 | 0.92 |

### Example 5

All BDix rats (185 g) are treated once with patupilone (PAT) at 1.5 mg/kg i.v. bolus on Day 0. Rats receive 7 mg/kg prednisolone (Predn) or the Predn-vehicle p.o. for 5 consecutive days (except G3 for 3 days) starting either on Day-0 (G1-G3) or after 24 hr (D+1) or 24 hr prior to PAT (D−1). Results show the mean±SEM for diarrhea, survival and body-weight (BW) using analyses described fully in Methods, where *P<0.05, **P<0.01, ***P<0.001 versus the control i.e. rats receiving patupilone alone with the prednisolone vehicle (one-way ANOVA with Tukey applied post-hoc).

| | | Host Response | | | | |
|---|---|---|---|---|---|---|
| | | Diarrhoea: | Diarrhoea: | | BW (g) | Δ% BW |
| Treatment | | AUC | Days > G1 | Survival | Day-7 | Day-7 |
| G1 | PAT & Veh-Predn | 4.5 ± 0.7 | 1.5 ± 0.3 | 8/8 | 163 ± 3 | −12.0 ± 1.2 |
| | (D0) | | | | | |
| G2 | PAT & 5 days | 0.1 ± 0.1*** | 0 ± 0* | 8/8 | 156 ± 4 | −15.9 ± 0.9 |
| | Predn (D0) | | | | | |
| G3 | PAT & 3-days | 0.5 ± 0.4*** | 0.1 ± 0.1 | 8/8 | 157 ± 3 | −14.9 ± 1.2 |
| | Predn (D0) | | | | | |
| G4 | PAT & 5-days | 0.8 ± 0.3*** | 0 ± 0* | 8/8 | 157 ± 3 | −15.1 ± 0.8 |
| | Predn (D−1) | | | | | |
| G5 | PAT & 5-days | 0.8 ± 0.4*** | 0 ± 0* | 8/8 | 151 ± 2 | −18.5 ± 1.2** |
| | Predn (D+1) | | | | | |

### Example 6

Results from the 4 different experiments are shown in which the dose of patupilone is always 1.5 mg/kg i.v. bolus on day 0 (D0). Diarrhea is assessed daily from day 3 to day 7 post patupilone treatment as Grade 0, 1, 2 or 3 to give the mean AUC days 3-7 or the mean number of days in which diarrhoea was graded >G1. The effect of prednisolone (Predn) is summarised as the amount of diarrhea in the treated group divided by the amount in the control (patupilone alone) group to give the T/C.

| | Predn Dose | | | Diarrhoea reduction | |
|---|---|---|---|---|---|
| | p.o. | | Schedule of | (T/C) | |
| Example | mg/kg | mg/m² | Predn treatment | AUC_{D3-D7} | >G1_{D3-D6} |
| 1 | 1 | 6 | 7-days from D0 | 0.85 | 0.81 |
| | 3 | 17 | 7-days from D0 | 0.30 | 0.13 |
| | 10 | 60 | 7-days from D0 | 0.20 | 0.06 |
| 2 | 3 | 17 | 7-days from D0 | 0.14 | 0 |
| | 10 | 60 | 7-days from DO | 0.01 | 0 |
| 3 | 7 | 40 | 5-days from D−1 | 0.61 | 0.65 |
| | 7 | 40 | 3-days from D0 | 0.60 | 0.57 |
| | 7 | 40 | 5-days from D+1 | 0.57 | 0.52 |
| | 7 | 40 | 7-days from D+3 | 0.75 | 0.57 |
| 5 | 7 | 40 | 5-days from D0 | 0.03 | 0 |
| | 7 | 40 | 3-days from D0 | 0.11 | 0.08 |
| | 7 | 40 | 5-days from D−1 | 0.18 | 0 |
| | 7 | 40 | 5-days from D+1 | 0.17 | 0 |

These Examples demonstrate that the corticosteroid prednisolone is able to reproducibly reduce diarrhea caused by an efficacious dose of patupilone in BDix rats. In 3 of 4 experiments this effect is dramatic and highly significant almost entirely abrogating diarrhea over the 4-5 days during which it is normally observed. The effect is dose-dependent and shows efficacy at doses that are commonly used in patients to treat some of the side effects of cancer chemotherapy, i.e. from 40-60 mg/m² (7-10 mg·kg in rats). The schedule of prednisolone does not appear to significantly impact efficacy, and treatment for 3 days is similarly effective to treatments for 5-7 days. There is no evidence that prednisolone interferred with significantly with patupilone exposure. Similar results are seen with prednisone and dexamethosone.

### Example 7

In human clinical trials, patients with brain metastases (from non small cell lung cancer) are treated with 4-8 mg p.o. (orally) of dexamethasone daily to control brain swelling. Thus, equivalent to a range of 28-128 mg/day prednisone (or prednisolone), i.e., 14-64 mg/m² per day of prednisone. The addition of glucocorticoids to patupilone seems to be reducing epothilone B (patupilone)-induced diarrhea.

### Example 8

HRPC (hormone-refractory prostate cancer) patients in a clinical trial are getting 10 mg (ca. 5.5 mg/m²) prednisone per day. This does not seem to have an effect on epothilone B (patupilone)-induced diarrhea.

1. A method of treatment for diarrhea associated with administering an epothilone derivative of formula (I) 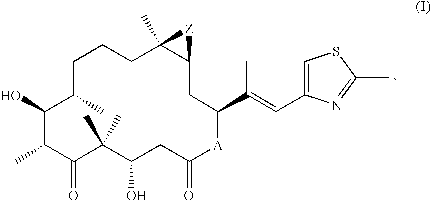
in which
compound A represents O or NR_{N}, wherein R_{N} is hydrogen or lower alkyl;
R is hydrogen or lower alkyl; and
Z is O or a bond, in free form or in the form of a pharmaceutically acceptable salt, to a patient in need thereof,
which comprises administering an effective amount of a corticosteriod antidiarrheal agent to the patient receiving treatment with the epothilone derivative.

2. A method according to claim 1, wherein the antidiarrheal agent is a glucocorticosteroid.

3. A method according to claim 1 wherein the antidiarrheal agent is a glucocorticosteroid selected from the group consisting of prednisone, prednisolone and dexamethosone.

4. The method of claim 1, wherein the epothilone derivative is epothilone B.

5. A pharmaceutical composition comprising a quantity which is jointly therapeutically effective against a proliferative disease of a combination according to claim 1 and at least one pharmaceutically acceptable carrier.

6. A method according to claim 3, wherein said glucocorticoid steriod is prednisolone.

7. A method according to claim 1, wherein said antidiarrheal agent is administered separately, sequentially or simultaneously with said epothilone derivative of formula (I) to a patient in need thereof.

8. A method according to claim 1, wherein said antidiarrheal agent is administered to a patient in need thereof once or twice per day.

9. A method according to claim 1, wherein said antidiarreal agent is administered in an amount of about 10 mg/m² per day to about 100 mg/m² per day to a patient in need thereof.

10. A method according to claim 9, wherein said antidiarrheal agent is administered to a patient in need thereof from about 3 days to about 7 days.

11. A method according to claim 9, wherein said antidiarreal agent is predinsolone.

12. Use of an epothilone or an epothilone derivative of formula (I) 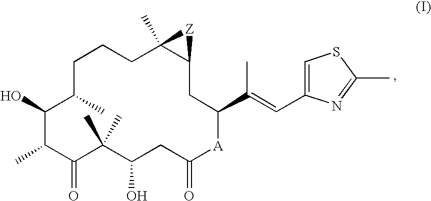
wherein
A represents O or NR_{N}, wherein R_{N} is hydrogen or lower alkyl;
R is hydrogen or lower alkyl; and
Z is O or a bond;
or a pharmaceutically acceptable salt for the preparation of a medicament for the treatment of a proliferative disease wherein the epothilone derivative is used in combination with a corticosteroid antidiarrheal agent for simultaneous, separate or sequential use.

13. Use according to claim 12, wherein the epothilone derivative is epothilone B.

14. Use according to claim 12, wherein the antidiarrheal agent is a corticosteroid.

15. Use according to claim 12, wherein the antidiarrheal agent is a glucocorticoid steroid selected from the group consisting of prednisone, prednisolone, dexamethosone.

16. Use according to claim 15, wherein the antidiarrheal agent is prednisolone.

17. Use of a combination according to claim 12, for the treatment of a proliferative disease.

18. Use of a combination according to claim 12, for the preparation of a medicament for the treatment of a proliferative disease.
